(19) United States
(12) Patent Application Publication
(10) Pub. No.: US 2026/0232484 A1
(43) Pub. Date: Aug. 13, 2026

(54) A THERMAL EYE LUBRICATION DEVICE

(71) Applicant: OCULAB PRIVATE LIMITED, Palghar, Maharashtra (IN)

(72) Inventors: Hemal KENIA, Mumbai, Maharashtra (IN); Paras SHAH, Mumbai, Maharashtra (IN); Sanandan SUDHIR, Ahmedabad, Gujarat (IN)

(21) Appl. No.: 19/151,846

(22) PCT Filed: May 3, 2024
(86) PCT No.: PCT/IB2024/054304

§ 371 Date: Jul. 29, 2025

(30) Foreign Application Priority Data
May 9, 2023 (IN) 202321032867

(51) Int. Cl. A61F 7/00; A61F 9/00

(52) U.S. Cl. CPC A61F 7/007; A61F 9/00; A61F 2007/0004; A61F 2007/0071; A61F 2007/0078

(57) ABSTRACT

An eye lubrication device includes a film-based heater sandwiched between a front foam and a back foam with a middle foam backing the heater. The front foam, the back foam, and the middle foam are thermoformed to create a contoured surface that snugly rests against the closed eyelids of the user. Film-based heater ensures uniform heating devoid of any hot spots and the corresponding side effects.

### TECHNICAL FIELD

The present disclosure relates to the field of devices for treatment of dry eye. In particular, the present disclosure relates to a thermal eye lubrication device.

### BACKGROUND

Background description includes information that may be useful in understanding the present invention. It is not an admission that any of the information provided herein is prior art or relevant to the presently claimed invention, or that any publication specifically or implicitly referenced is prior art.

Incidence of dry eye disease is becoming more common on account of long digital screen exposure. A study by All India Institute Of Medical Sciences (AIIMS), New Delhi, suggests that 32% Indians, of which 90% are computer users, suffer from Dry Eye.

Dry Eye is a multifactorial disease of the tears and the ocular surface that results in symptoms of discomfort, visual disturbance and tear-film instability with potential damage to the ocular surface. Digital screen exposure is one of the most common risk factors associated with Dry Eye.

Apart from eyes, dry eye also impacts the quality of life, mental health and psychological wellbeing. Besides, it is a significant global economic burden and causes substantial loss in work productivity.

In healthy eyes, Meibomian tear glands located under the upper and lower eyelid, secrete the lipid component (‘meibum’) of tears, which helps form a ‘stable’ tear-film. A stable tear-film moisturizes and nourishes the eye surface, and helps prevent symptoms and signs of Dry Eye. However, the digital screen exposure, along with infrequent and incomplete blinking, causes the meibum inside the Meibomian tear glands to solidify, leading to ‘Meibomian Gland Dysfunction’. Solidified meibum clog the opening and lumen of dysfunctional glands, and later, permanently destroy them. Absence of meibum leads to ‘unstable’ tear-film. This causes excessive tear evaporation, leading to ‘Evaporative’ Dry Eye.

A primary care recommended for Meibomian Gland Dysfunction related evaporative dry eye is eyelid warming. Traditional eyelid warming is done manually with cloth or cotton soaked in warm water. However, when done manually, it is difficult to maintain the required temperature and pressure necessary for safety and predictable clinical outcomes. Also, it is cumbersome and time-consuming, leading to poor compliance.

Considering the sheer volume and impact of Meibomian Gland Dysfunction related Evaporative Dry Eye, it is imperative to develop a predictable and standardized, yet simple and easy solution for the same. Electric warming eye compress is one such medical device. When worn snugly on shut eyelids, it raises the temperature of underlying Meibomian tear glands to a preset level. This enables liquefaction of the solidified meibum to unclog the Meibomian tear glands, and preserve their health. This ‘liquified’ meibum flows onto the eye surface and forms a ‘stable’ tear-film. A stable and healthy tear film helps relieve the symptoms and signs related to Evaporative Dry Eye.

However, the existing warming eye compresses have poor contact on lower eyelid skin and corners of both upper and lower eyelid skin. Hence, efficacy of these devices is sub-optimal on those Meibomian tear glands that are located underneath these areas. Also, safety remains a concern with most such devices, as they draw dangerously high electrical energy to generate therapeutically effective heat. On the other side, those devices that draw low electrical energy, heat generated is often clinically ineffective.

These drawbacks in the known warming eye compresses necessitate the development of an improved device that overcomes them.

Therefore, there is a requirement of an improved eye lubrication that overcomes the drawbacks of the conventional eye devices.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

### OBJECTS OF THE INVENTION

A general object of the present disclosure is to overcome drawbacks of the conventional warming eye compresses.

An object of the present disclosure is to provide an eye lubrication device that is safe to use.

Another object of the present disclosure is to provide an eye lubrication device that draws less power.

Another object of the present disclosure is to provide an eye lubrication device that has low thermal loss.

Another object of the present disclosure is to provide an eye lubrication device that provides a Uniform heating of eye all across its surface.

Another object of the present disclosure is to provide an eye lubrication device that provides therapeutically optimal temperatures across all points on upper and lower eyelid.

Another object of the present disclosure is to provide an eye lubrication device that eliminates ‘hot-pockets’ induced localized skin burns or cicatrizing conjunctivitis.

Another object of the present disclosure is to provide an eye lubrication device that allows different modes of heating.

Another object of the present disclosure is to provide an eye lubrication device that can be operated remotely through a mobile device.

### SUMMARY

Aspects of the present disclosure relate generally to devices for treatment of dry eye. In particular, the present disclosure relates to a simple, compact, and efficient thermal eye lubrication device.

According to an aspect, a thermal eye lubrication device includes an elongate strap. The elongated strap includes a back foam and a front foam. The back foam and the front foam are thermoformed to a contoured shape in an eye portion of the elongate strap. The contoured shape is configured to allow a snug fit over the closed eyes of a user. The device in addition includes a heater. The heater is configured in the eye portion. The heater provides a warm compress to the eyes of the user to heat the Meibomian tear glands of the eyes to unclog the Meibomian tear glands for lubricating the eyes of the user. Moreover, the heater is a film based heater to provide uniform heating to the eyes of the user.

In an embodiment, the device may include an interface portion. The interface portion may include USB port. The interface portion may couple the device to a power source.

In an embodiment, the interface portion may comprises a button to activate the device, wherein the button may be configured to enable operating the device in different modes of operation.

In an embodiment, the different modes of operation of the device may include different temperature levels and different duration of heating.

In an embodiment, the interface portion may include a pre coded epoxy sealed PCB. The PCB may be coupled to the heater.

In an embodiment, the device may include a control unit. The control unit may be in communication with a mobile device. The control unit may be configured within the device. The control unit may operate the device remotely upon receiving the inputs from the user of the device through the mobile device.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

FIGS. 1A and 1B illustrate a front and a rear view respectively of the disclosed eye lubrication device, in accordance with embodiments of the present disclosure.

FIG. 2 illustrates the disclosed eye lubrication device of FIGS. 1A and 1B being used by a user, in accordance with embodiments of the present disclosure.

FIG. 3 illustrates an exemplary exploded view of the eye lubrication device of FIGS. 1A and 1B, in accordance with embodiments of the present disclosure.

FIG. 4 illustrates a network diagram of the eye lubrication device 100 in communication with a mobile device 400, in accordance with embodiments of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims.

Various terms are used herein. To the extent a term used in a claim is not defined, it should be given the broadest definition persons in the pertinent art have given that term as reflected in printed publications and issued patents at the time of filing.

Existing warming eye compresses use wire-based heater. Their drawbacks include high thermal loss which leads to low rise of eye compress surface temperature to therapeutically effective levels, drawing high current from 2 or 3 pin sockets to attain therapeutically effective temperature, do not attain therapeutically effective temperature, when drawing low current from USB-powered sockets. They also lead to non-uniform heat distribution, leading to extremely high heating of eye compress surface placed just outside the wire-based heater, causing possibility of localized skin burns and cicatrizing conjunctivitis, poor heating of eye compress surface placed away from the wire-based heater causing multiple pockets of therapeutically sub-optimal temperatures

Furthermore, flat surface of existing warming eye compresses is a complete misfit for the curved eye contour. In a shut eye, existing devices fail to deliver therapeutically effective heat to meibomian glands located under the lower eyelid and those located under upper and lower eyelid corners. This is due to the larger size and forward bulging contour of the upper eyelid. In order to optimally touch the eyelid corners and lower eyelid, existing warming eye compresses have to be placed tightly over the eyes. Placing compress tightly is not only extremely uncomfortable but also potentially dangerous, as it causes collapse of the Kessing's space 11 under the tarsus, as well as collapse of eyelid and palpebral conjunctival blood vessels. This not only brings the warming eye compress dangerously close to the cornea and other inner eye structures, but also impedes the cooling of these heated intra-ocular structures (in the form of heat transfer by flowing away of warmed aqueous humor, limbal blood and tear-film. This causes ocular globe compression and excessive heating of the inner eye structures, both of which lead to short-term side-effects and long-term complications, as under:

a) Ocular globe compression with the short-term complication of inducing polygonal reflex of Fischer-Schweitzer, causing transient visual blur, and long-term complication resulting in frequent spikes in the intraocular pressure, leading to Glaucoma-related Optic nerve damage and permanent vision loss.
b) Excessive heating of inner eye structures with the short-term complications of ‘Softening’ of cornea leading to transient visual blur, and the long-term complications of thermal injury to crystalline lens leading to early-onset cataract and thermal injury to the cornea that reduces its bio-mechanical strength, leading to corneal molding (‘warpage’) and keratoconus.

The short-term side-effects and potential long-term complications of existing warming eye compress are summarized in the table below:

| Mechanism | Short-term Side-effects | Long-term Complications |
|---|---|---|
| a) Ocular globe | Transient visual blur | Glaucoma-related Optic |
| compression | | nerve damage and |
| | | permanent vision loss |
| b) Heating of | Transient visual blur | Cataract |
| intra-ocular | | Corneal molding/warpage |
| structures | | & Keratoconus |

In an aspect, the present disclosure provides a device for warming eye compresses that overcomes the above drawbacks. In an aspect, the disclosed device includes a film based heater, which leads to extremely low thermal loss and resultant quick rise of eye compress surface temperature to therapeutically effective levels, drawing low (<0.5 Ampere) current from USB-powered sockets, and yet attaining therapeutically effective temperatures. Further, a fixed internal resistance and voltage of heater limits total power and prevents drawing of excessive current, and thereby overheating. The film based heater also results in an uniform heat distribution with the resultant uniform warm heating of eye compress all across its surface, which provides therapeutically optimal temperatures across all points on upper and lower eyelid, while eliminating ‘hot-pockets’ induced localized skin burns or cicatrizing conjunctivitis.

In another aspect, the disclosed warming eye compress device includes a contoured surface derived from 3D mapping of user eye and facial contours, which forms a perfect fit for most users, thereby, allowing uniform, therapeutically effective heat transfer to all meibomian glands of both upper as well as lower eyelids. This helps liquefy and release meibum from all diseased glands of upper and lower eyelids, to form a stable, high-quality tear film.

In another aspect, the disclosed device, by virtue of its unique contoured surface, the device does not have to be placed tightly over the eyes. Placing compress gently itself is enough to maintain contact over all points across both upper and lower eyelids. The leads to advantages of non-collapse of the Kessing's space under the tarsus as well as eyelid and palpebral conjunctival blood vessels. It also keeps the device at a safe distance away from the inner eye structures, but also quickens the cooling of intra-ocular structures (in the form of heat transfer by ‘warmed’ blood, flowing away from the eye).

In another aspect, the disclosed device attains therapeutically effective temperature in both upper and lower eyelid meibomian glands, by simply gently placing it on the shut eye, without having the need to tighten it at all. This completely eliminates the eye ocular globe compression and excessive heating of the inner eye structures, and with it, all possible short-term side-effects and long-term complications of existing eye compresses as mentioned before.

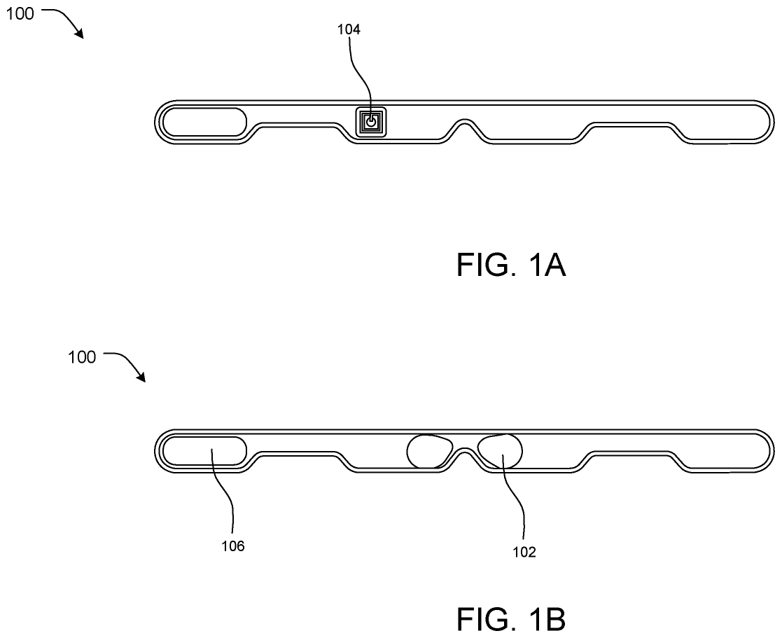

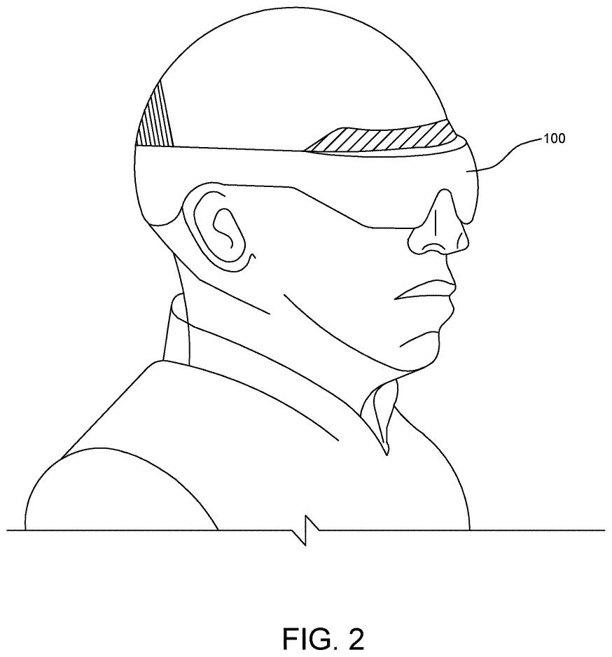

Referring now to FIGS. 1A and 1B, where front and rear views of the disclosed eye lubrication device are shown, the device 100 can be an elongate strapped shaped device having an eye portion 102 located in the middle of the elongate strap such that when wound around head of a user, as shown in FIG. 2, the eye portion 102 sits over the closed eyelids of the user. The eye portion can include a contoured surface to snugly fit over the closed eyes and further includes a heater to heat the eye portion 102. The contoured surface can be achieved through thermoforming or any other forming method.

The strap portion can include Velcro 106 at the ends to enable fastening of the eye lubrication device 100 around the head as shown in FIG. 2. Moreover, the device 100 can be worn on the nose or can be held in the user's hand like a magnifying glass with a handle besides wearing the device like normal spectacles.

The eye lubrication device 100 can further include an interface portion 104 that can facilitate the eye lubrication device 100 to be coupled to a power source, to actuate the eye lubrication device 100 under different modes and to indicate operating state of the eye lubrication device 100.

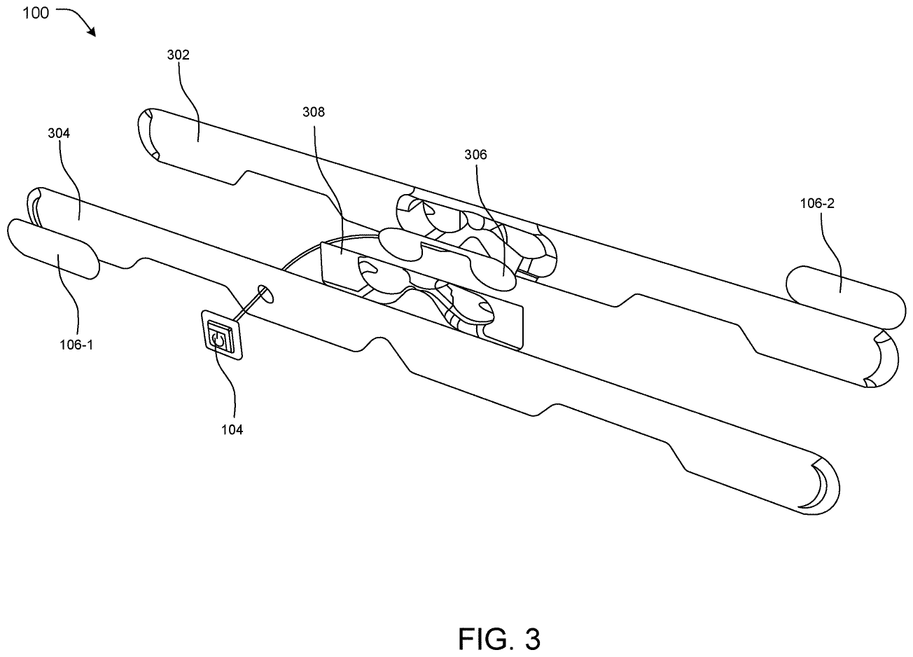

FIG. 3 shows an exploded view of the eye lubrication device 100 and as shown therein, the eye lubrication device 100 can include a back foam 302 and a front foam 304, each thermoformed and together forming the elongate strap portion of the eye lubrication device 100. The thermoforming can give the strap portion a contoured shape in the eye portion 102. The eye portion 102 can accommodate a heater 306, which can be a film-based heater for uniform heating of the eye portion 102, There can be a middle foam 308 again thermoformed to give a matching contoured shape. A pair of Velcro pieces, such as 106-1 and 106-2 can be provided on opposite sides of the eye lubrication device 100 at the two ends of the strap portion.

The interface portion 104 can include a button to activate/deactivate the eye lubrication device 100, such as a long pressing of the button can activate the eye lubrication device 100. The interface portion 104 can include a USB port to connect the device 100 to a power source, and indication lights to indicate status of the device 100. The interface portion 104 can have a pre coded epoxy sealed PCB inside with 3 cables connecting the interface portion 104 to the heater 306. In an aspect, the device 100 can be operated in different modes, such as at different temperature levels and different durations, such as 5 minutes, 10 minutes, and 15 minutes, depending on the type of therapy required. In an exemplary embodiment, a battery electrically coupled to the device can supply electricity to the device through an USB port. Yet in another embodiment, any external device that can generate electricity can be electrically coupled to the USB port to supply electricity to the device.

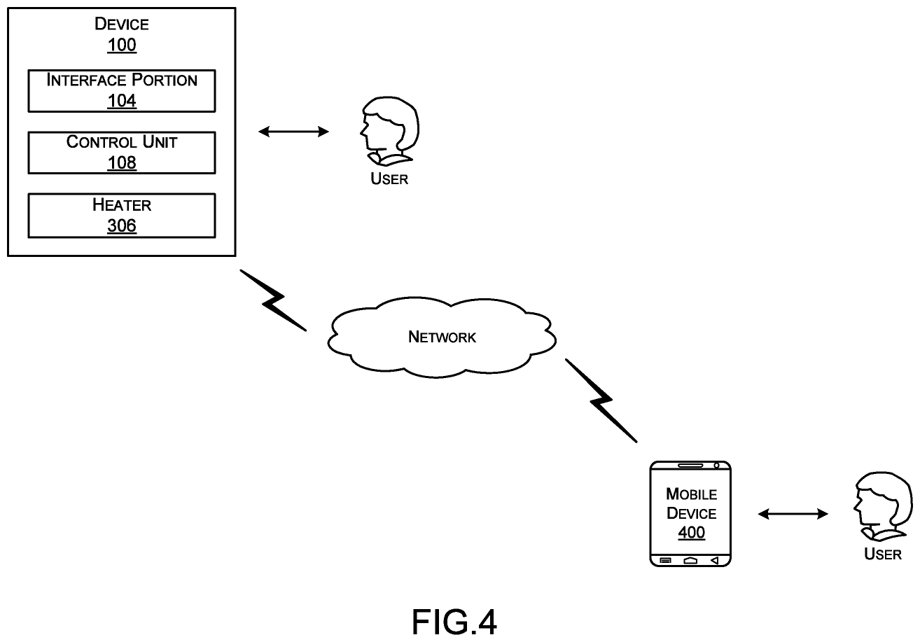

FIG. 4 shows a network diagram of the eye lubrication device 100 in communication with a mobile device 400 and as shown therein, where the eye lubrication device 100 can include a control unit 108 in communication with a mobile device 400, configured within the device 100 for operating the device 100 remotely upon receiving the inputs from the user of the device 100 through the mobile device 400. In an exemplary embodiment, the device 100 can be integrated with the mobile device 400 such that a dedicated mobile application of the mobile device 400 can actuate the device 100 at the instance of the use of the device 100. The user's data stored in the mobile application can aid in customizing and further improving the functionality of the device 100.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### Advantages of the Invention

The present disclosure provides an improved eye lubrication device that overcomes drawbacks of the conventional eye lubrication devices.

The present disclosure provides an eye lubrication device that is safe to use.

The present disclosure provides an eye lubrication device that draws less power.

The present disclosure provides an eye lubrication device that has low thermal loss.

The present disclosure provides an eye lubrication device that provides a Uniform heating of eye all across its surface.

The present disclosure provides an eye lubrication device that provides therapeutically optimal temperatures across all points on the upper and lower eyelid.

The present disclosure provides an eye lubrication device that eliminates ‘hot-pockets’ induced localized skin burns or cicatrizing conjunctivitis.

The present disclosure provides an eye lubrication device that allows different modes of heating.

The present disclosure provides an eye lubrication device that can be operated remotely through a mobile device.

1. A device for lubricating eyes, comprising:
an elongate strap comprising a back foam and a front foam;
each of the back foam and the front foam thermoformed to a contoured shape in an eye portion of the elongate strap with a middle form, the contoured shape being configured to allow a snug fit over closed eyes of a user; and,
a heater configured in the eye portion to provide a warm compress to the eyes of the user to heat Meibomian tear glands of the eyes, thereby unclogging the Meibomian tear glands for lubrication of the eyes; wherein the heater is a film based heater to provide uniform heating to the eyes of the user.

2. The device as claimed in claim 1, wherein the device comprises an interface portion, the interface portion comprising a USB port for coupling the device to a power source.

3. The device as claimed in claim 1, wherein the interface portion comprises a button to activate the device, wherein the button is configured to enable operating the device in different modes of operation.

4. The device as claimed in claim 1, wherein the different modes of operation comprise different temperature levels and different duration of heating.

5. The device as claimed in claim 1, wherein the interface portion comprises a pre-coded epoxy sealed PCB that is coupled to the heater.

6. The device as claimed in claim 1, wherein the device comprises a control unit in communication with a mobile device, configured within the device for operating the device remotely upon receiving the inputs from the user of the device through the mobile device.
